(19) United States
(12) Patent Application Publication
(10) Pub. No.: US 2025/0017519 A1
(43) Pub. Date: Jan. 16, 2025

(54) BIOLOGICAL EXAMINATION APPARATUS, BIOLOGICAL-INFORMATION ANALYSIS METHOD, AND COMPUTER PROGRAM

(71) Applicant: Maxell, Ltd., Kyoto (JP)

(72) Inventors: Yoshiharu UCHIDA, Kyoto (JP); Ran TACHIBANA, Kyoto (JP); Tomohiko MIZUGUCHI, Kyoto (JP)

(73) Assignee: Maxell, Ltd., Kyoto (JP)

(21) Appl. No.: 18/714,570

(22) PCT Filed: Dec. 3, 2021
(86) PCT No.: PCT/JP2021/044466

§ 371 Date: May 30, 2024

(51) Int. Cl. A61B 5/00; A61B 5/05; A61B 5/11

(52) U.S. Cl. CPC A61B 5/4205; A61B 5/05; A61B 5/1126; A61B 5/7235; A61B 5/742; A61B 2562/0204

(57) ABSTRACT

A biological examination apparatus extracts an up-and-down movement component associated with the thyroid cartilage and a back-and-forth movement component associated with the thyroid cartilage from a fitting result obtained by fitting a model function modeling the swallowing movement to distance information based on detection data detected by the larynx portion displacement detector. Based on the extracted components, two-dimensional trajectory data indicating the movement trajectory of the thyroid cartilage is generated. In addition, based on data from a swallowing sound detector, identification display data to determine, on the trajectory graph, which of the outbound path of the movement path according to the elevation and the forward movement of the thyroid cartilage and the return path of the movement path according to the movement of the thyroid cartilage has a peak of the swallowing sound, in a series movement paths of the thyroid cartilage at the time of swallowing, is generated.

### TECHNICAL FIELD

The present invention relates to a biological examination apparatus for performing an examination related to swallowing of a living body, a biological information analysis method for analyzing biological information obtained with the swallowing of the living body, and a computer program.

### BACKGROUND ART

Pneumonia is known as one of the major causes of death. Among these, aspiration pneumonia induced by dysphagia, which means a disorder related to swallowing, accounts for about 60% or more.

The main causative disease of dysphagia is stroke, and it is known that dysphagia occurs in 80% of acute phase patients. In addition, it is also known that the proportion of dysphagia increases as the age increases even without a clear causative disease such as stroke, and in an aging society, it is expected that aspiration pneumonia and dysphagia will increase in the future.

Therefore, various tests for diagnosing dysphagia have been conventionally attempted. For example, swallowing enhancement (Videofluoroscopic Examination of Swallowing: VF) is generally known as a method for accurately evaluating and grasping dysphagia. In this VF, the movement of the alimentary mass at the time of swallowing in the subject and the movement of the lingual bone or the larynx are monitored using an alimentary mass containing a contrast medium such as barium sulfate and an X-ray fluoroscopic apparatus. In this case, the swallowing movement is a series of quick movements and is therefore generally recorded in a video and evaluated. However, VF requires attention because it is a test that potentially has a possibility of aspiration, asphyxiation, or the like, and also has problems such as exposure, time restriction, and high cost because an X-ray fluoroscopic apparatus that is a large apparatus is required. For example, swallowing enhancement (Videoendoscopic Examination of Swallowing: VE) is generally known as a method for accurately evaluating and grasping dysphagia, but has the same problems as those of VF. As described above, a clinical test such as VF or VE can be accurately diagnosed because the movement of the throat is directly observed, but the clinical test is highly invasive and requires predetermined equipment and thus cannot be easily performed anywhere.

On the other hand, as a simple test method for dysphagia, a screening test such as palpation (Repetitive Saliva Swallowing Test (RSST)), auscultation (cervical auscultation method), observation (water drinking test and food test), or subjective evaluation by a questionnaire is known. However, there is a problem that quantitative evaluation is difficult and reproducibility and objectivity are poor although the screening test can be performed as a daily test.

In view of the above problems, in recent years, some methods for sharing and recording the swallowing state have been proposed. For example, Patent Literature 1 discloses an apparatus that attaches a microphone to the neck, stores audio data corresponding to auscultation as digital data, and detects swallowing by waveform analysis. In addition, Patent Literature 2 discloses a biological examination apparatus in which a magnetic coil is attached to the neck in addition to a microphone, movement data of the thyroid cartilage at the time of swallowing corresponding to palpation is stored as digital data in addition to audio data, and an examination regarding swallowing of a living body and a result of the examination are displayed. Specifically, by arranging the transmission coil and the reception coil so that the thyroid cartilage is interposed therebetween, the biological examination apparatus measures, as distance information between the coils, the displacement in the horizontal direction of the thyroid cartilage caused in association with the two-dimensional movement of the hyoid in its up-and-down and back-and-forth directions at the time of swallowing. According to such an examination form, since distance information and audio information corresponding to palpation and auscultation can be acquired simultaneously and non-invasively, the distance information and the audio information can be combined to evaluate the swallowing movement.

### CITATION LIST

### Patent Literature

Patent Literature 1: JP 2013 017694 A
Patent Literature 2: JP 2009-213592 A

### SUMMARY OF INVENTION

### Technical Problem

Incidentally, in the biological examination apparatus of Patent Literature 2 described above, the distance information and the audio information are independently displayed as time-series waveforms. Therefore, the evaluation of the swallowing state is performed by comparing two types of waveforms, that is, the movement waveform based on the distance information and the swallowing sound waveform based on the audio information with respect to the timing of the temporal change. However, in particular, the movement waveform based on the distance information is a result of indirectly observing the movement of the hyoid through the thyroid cartilage, and the two-dimensional movement of the thyroid cartilage in its up-and-down and back-and-forth directions is indirectly assumed as the one-dimensional movement of the left and right. Therefore, it is difficult to interpret actual swallowing dynamics from the time-series waveform, and the examiner has to estimate the comprehensive swallowing movement from the waveform change of the audio information and the distance information. In such an evaluation form based on display of two independent time-series waveforms, there is a problem that it is difficult to specifically grasp at a glance how the swallowing movement is.

In addition, when accurately grasping the swallowing movement, it is also important to determine whether there is a peak of the swallowing sound in the outbound path of the movement path according to elevation and forward movement of the thyroid cartilage (the path when the food bolus, saliva, or the like is swallowed and passes through the esophagus) or the return path of the movement path according to backward movement and descent of the thyroid cartilage (the path when the food bolus, saliva, or the like completely passes through the esophagus and is sent into the stomach) in a series of movement paths of the thyroid cartilage in its up-and-down and back-and-forth directions at the time of swallowing. However, in the conventional technology including Patent Literatures 1 and 2, it is not possible to easily grasp the peak position of the swallowing sound in such a thyroid cartilage movement path at the time of swallowing.

The present invention has been made in view of the above circumstances, and an object of the present invention is to provide a biological examination apparatus, a biological information analysis method, and a computer program that enable two-dimensional movements in up-and-down and back-and-forth directions of the thyroid cartilage and the hyoid bone accompanied by the swallowing sound to be grasped at a glance as the swallowing dynamics by a non-invasive examination and enable a peak position of the swallowing sound in the thyroid cartilage movement path at the time of swallowing to be grasped at a glance.

### Solution to Problem

In order to solve the above problems, a biological examination apparatus of the present invention includes: a larynx portion displacement detector that detects a change in a distance between two positions in a larynx portion of a subject caused by movement of a thyroid cartilage in an up-and-down direction and a back-and-forth direction at the time of swallowing; a swallowing sound detector that detects a swallowing sound when the subject swallows; and a processor that processes detection data from the larynx portion displacement detector and the swallowing sound detector. The processor extracts an up-and-down movement component associated with an up-and-down movement of the thyroid cartilage and a back-and-forth movement component associated with a back-and-forth movement of the thyroid cartilage from a fitting result of fitting a model function modeling a swallowing movement to distance information based on the detection data detected by the larynx portion displacement detector and generates two-dimensional or three-dimensional trajectory data, which indicates the movement of the thyroid cartilage in the up-and-down direction and the back-and-forth direction simultaneously in one trajectory graph, based on the extracted up-and-down movement component and back-and-forth movement component, and generates identification display data that makes it possible to identify, on the trajectory graph, which of an outbound path of a movement path according to elevation and forward movement of the thyroid cartilage and a return path of the movement path according to a backward movement and a downward movement of the thyroid cartilage has a peak of swallowing sound in a series of back-and-forth and up-and-down movement paths of the thyroid cartilage at the time of swallowing, based on the detection data from the swallowing sound detector.

For the distance information based on the detection data, that is, a W-shaped distance waveform 701 (the horizontal axis indicates time and the vertical axis indicates a distance between two positions.) indicating a temporal change in the distance between two positions in the larynx portion of the subject caused by the movement in the up-and-down direction and the back-and-forth direction of the thyroid cartilage at the time of swallowing, illustrated as an example in FIG. 11, the present inventors have recognized that two-dimensional movements (back-and-forth movement and up-down movement) of the thyroid cartilage and the lingual bone are embedded in a one-dimensional (left and right) space due to the pyramidal shape of the thyroid cartilage, and have modified the way of grasping the components in the distance waveform 701 to a unique way different from the conventional way of grasping, thereby finding an innovative information presentation form that can at a glance grasp the two-dimensional movements of the up-and-down and back-and-forth of the thyroid cartilage and the lingual bone accompanied by the swallowing sound as the swallowing dynamics. Specifically, in the distance waveform 701, a downwardly convex waveform component is generated in a series of movements from rising to falling of the thyroid cartilage, and an upwardly convex waveform component is generated in a series of movements from advancing to reversing of the thyroid cartilage. However, the present inventors have found that this distance waveform 701 of the W type is different from a conventional technique in which a combination of a downwardly convex waveform 710a, an upwardly convex waveform 720, and a downwardly convex waveform 710b is captured as illustrated in FIG. 11 (a), and a fitting result obtained by fitting a model function modeling the swallowing movement to the distance waveform 701 is obtained by regarding superposition of a gently downwardly convex waveform 710 and a sharp upwardly convex waveform 720 as illustrated in FIG. 11 (b). In addition, a biological information analysis form has been found that extracts a back-and-forth movement component associated with the back-and-forth movement of the thyroid cartilage corresponding to the upwardly convex waveform 720 and an up-and-down movement component associated with the up-and-down movement of the thyroid cartilage corresponding to the downwardly convex waveform 710 and generates two-dimensional trajectory data indicating movement trajectories in the up-and-down direction and the back-and-forth direction of the thyroid cartilage based on the extracted up-and-down movement component and the back-and-forth movement component.

According to the above configuration of the present invention, since the fitting result is obtained by fitting the model function modeling the swallowing movement to the distance information based on the detection data detected by the larynx portion displacement detector, the movement of the thyroid cartilage (lingual bone) can be reproduced two-dimensionally in a non-invasive manner (modeling of the swallowing movement), and movement components related to all the movement directions of the thyroid cartilage at the time of swallowing, that is, two back-and-forth movement components and up-and-down movement components respectively corresponding to the movement in the up-and-down direction and the back-and-forth direction are extracted from the fitting result, and two-dimensional or three-dimensional trajectory data indicating the movement in the up-and-down direction and the back-and-forth direction of the thyroid cartilage simultaneously in one trajectory graph is generated based on these two components. Therefore, as in Patent Literature 2 described above, it is also possible to grasp, at a glance, up-and-down and back-and-forth movements of the thyroid cartilage (lingual bone) as two-dimensional or three-dimensional swallowing dynamics without requiring estimation of comprehensive swallowing movement (grasp at a glance how the swallowing movement is specifically). That is, according to the present invention, the swallowing dynamics including two pieces of physical information (up-and-down movement information and back-and-forth movement information of the thyroid cartilage) can be integrated into one trajectory graph and visualized by modeling and component decomposition of the swallowing movement. Therefore, the up-and-down and back-and-forth movements of the thyroid cartilage (lingual bone) can be two-dimensionally or three-dimensionally grasped at a glance. As a result, it is possible to easily evaluate dysphagia without requiring skill.

In this case, it is preferable that the two-dimensional trajectory data is generated as coordinate data indicated on a coordinate plane defined by two coordinate axes perpendicular to each other and that one of the coordinate axes corresponds to a trajectory data value of the back-and-forth movement component and the other coordinate axis corresponds to a trajectory data value of the up-and-down movement component. In addition, it is preferable that the three-dimensional trajectory data is generated as coordinate data indicated in a coordinate space defined by three coordinate axes perpendicular to each other and the three coordinate axes include a coordinate axis corresponding to a trajectory data value of the back-and-forth movement component, a coordinate axis corresponding to a trajectory data value of the up-and-down movement component, and a coordinate axis indicating a swallowing movement time. In fact, it has been confirmed by the present inventors that the display form based on such trajectory data values approximately corresponds to the movement trajectory of the lingual bone in the swallowing dynamics analysis such as the lingual bone movement by the videofluoroscopic examination of swallowing (VF).

In addition to the above, in the above configuration of the present invention, it is possible to identify by identification display, on the trajectory graph, which of the outbound path of the movement path according to the elevation and the forward movement of the thyroid cartilage and the return path of the movement path according to the backward movement and the downward movement of the thyroid cartilage has a peak of the swallowing sound in a series of up-and-down and back-and-forth movement paths of the thyroid cartilage at the time of swallowing. Therefore, the peak position of the swallowing sound in the thyroid cartilage movement path at the time of swallowing can be grasped at a glance, and the swallowing movement can be accurately evaluated. In addition, such identification display is useful particularly in a case where it is difficult to grasp which one of the outbound path and the return path of the thyroid cartilage movement path has the peak of the swallowing sound because dots of data plotted on the graph overlap each other, such as a case where the peak position of the swallowing sound is a position close to the coordinate origin in the two-dimensional trajectory graph display (two-dimensional still image) based on the two-dimensional trajectory data. In addition, in the two-dimensional trajectory graph display based on the two-dimensional trajectory data, it may be difficult to intuitively recognize in which direction the trajectory has progressed. Therefore, it is preferable that the processor generates reference display data for displaying reference information indicating a transition direction (progress direction) of the trajectory graph together with the trajectory graph. Examples of such reference display data include data for displaying an icon indicating the transition direction of the trajectory graph, data for displaying the progress of the trajectory graph in a moving image (animation), and the like.

In the above configuration, the “identification display” includes display in which a plot (mark) indicating the peak position of the swallowing sound is distinguished from plots of other data values by marks or characters such as color, size, and arrow, or display in which the peak position of the swallowing sound is indicated by dots on the trajectory graph in a state in which the trajectory graph is color-coded for the outbound path and the return path. In short, any display form may be used as long as a display form in which it is possible to grasp at a glance whether the peak of the swallowing sound is present in the outbound path or the return path of the thyroid cartilage movement path is used. In addition, the larynx portion displacement detector may adopt any detection form as long as the change in the distance between the two positions in the larynx portion of the subject caused by the movement in the up-and-down direction and the back-and-forth direction of the thyroid cartilage at the time of swallowing can be detected. For example, the larynx portion displacement detector may include a transmission coil and a reception coil that are arranged so as to interpose the thyroid cartilage from both sides and transmit and receive a high frequency signal, or may detect a change in the distance by three-dimensionally capturing the larynx portion (thyroid cartilage) with a stereo camera or the like and analyzing the image data.

In addition, in the above configuration, the processor may generate two-dimensional trajectory data individually indicating the temporary movement trajectory in each of the up-and-down direction and the back-and-forth direction of the thyroid cartilage based on the up-and-down movement component and the back-and-forth movement component. According to this, it is also possible to individually grasp the trajectories of the up-and-down movement and the back-and-forth movement of the thyroid cartilage, which can also contribute to detailed analysis of the swallowing movement.

In addition, in the above configuration, the processor may generate a swallowing sound waveform indicating a temporal change in the amplitude of the swallowing sound based on the detection data detected by the swallowing sound detector and generate identification display data for identifying and displaying a plot of each trajectory data value on the trajectory graph according to the magnitude of the amplitude of the swallowing sound by temporally associating the swallowing sound waveform and the trajectory graph with each other.

According to this, the movement of the larynx portion and the change in the swallowing sound can be integrated into one trajectory graph and visualized based on the two pieces of physical information (distance information and audio information) obtained from the larynx portion displacement detector and the swallowing sound detector. Therefore, the swallowing dynamics such as the timing of the swallowing movement and the swallowing sound can be non-invasively recognized at a glance. In addition, since the plot of each trajectory data value on the trajectory graph is identified and displayed according to the magnitude of the amplitude of the swallowing sound, it is possible to visually recognize at which timing the swallowing sound is emitted at a glance and to determine at which timing the substance put in the mouth is fed from the esophagus to the stomach at a glance.

In the above configuration, the “identification display” may be in any display form as long as the trajectory data values having different amplitudes of the swallowing sound can be identified by displaying the plots of the trajectory data values differently according to the magnitude of the amplitude of the swallowing sound, changing the magnitude or shape of the plot (mark) of the trajectory data values according to the magnitude of the amplitude of the swallowing sound, or the like.

In the above configuration, the processor may generate supplementary display data for displaying supplementary information, which includes a predetermined feature point associated with the fitting result, a predetermined feature point associated with the swallowing sound waveform, and an occurrence time of a trajectory data value plotted on the trajectory graph, to be superimposed on the trajectory graph. According to this, the trajectory graph display can be complemented by the supplementary information related to the movement of the larynx portion and the change in the swallowing sound, and the amount of information that can be read from the trajectory graph can be increased. Therefore, the evaluation of dysphagia can be performed more accurately and quickly. Examples of the “feature point” include a singular point and an inflection point in the waveform including the fitting result (for example, the fitted movement waveform) and the upper limit peak value and the lower limit peak value of the swallowing sound waveform or the waveform related thereto.

In the above configuration, the processor may generate reference display data for displaying reference information indicating a predetermined feature amount calculated from the trajectory graph together with the trajectory graph. According to this, information that is difficult to grasp only from the trajectory graph can be added and displayed together with the trajectory graph. Therefore, the degree of understanding of the trajectory graph can be enhanced, and it is possible to contribute to accurate and quick evaluation of dysphagia. In addition, examples of the “feature amount” include a maximum amount of displacement in the back-and-forth direction of the thyroid cartilage, a maximum amount of displacement in the up-down direction, a time difference between times when the movement waveform and the swallowing sound waveform take maximum values, and a ratio of the time difference to a variance value of displacement in the back-and-forth direction of the thyroid cartilage.

The present invention also provides a biological information analysis method and a computer program having the above-described characteristics. According to such a biological information analysis method and computer program, it is possible to obtain the same effects as those of the above-described biological examination apparatus.

### Advantageous Effects of Invention

According to the present invention, the up-and-down movement component associated with the up-and-down movement of the thyroid cartilage and the back-and-forth movement component associated with the back-and-forth movement of the thyroid cartilage are extracted from the fitting result obtained by fitting the model function modeling the swallowing movement to distance information based on detection data detected by the larynx portion displacement detector. Based on the extracted up-and-down movement component and back-and-forth movement component, two-dimensional trajectory data indicating the movement trajectory of the thyroid cartilage in the up-and-down direction and the back-and-forth direction is generated. Therefore, the two-dimensional movements in the up-and-down and back-and-forth of the thyroid cartilage and the lingual bone accompanied by the swallowing sound can be grasped at a glance as the dynamics of swallowing by the non-invasive examination. In addition, it is possible to identify by identification display, on the trajectory graph, which of the outbound path of the movement path according to the elevation and the forward movement of the thyroid cartilage and the return path of the movement path according to the backward movement and the downward movement of the thyroid cartilage has a peak of the swallowing sound in a series of up-and-down and back-and-forth movement paths of the thyroid cartilage at the time of swallowing. Therefore, the position of the peak of the swallowing sound in the thyroid cartilage movement path at the time of swallowing can be grasped at a glance, and the swallowing movement can be accurately evaluated.

### BRIEF DESCRIPTION OF DRAWINGS

FIG. 1 is a functional block diagram of a biological examination apparatus according to an embodiment of the present invention.

FIG. 2 is a schematic perspective view of a flexible holder that holds a larynx portion displacement detector of the biological examination apparatus in FIG. 1.

FIG. 3 is a functional block diagram of a calculator of the biological examination apparatus in FIG. 1.

FIG. 4 is a flowchart illustrating a flow of processing of a movement analyzer of a processor of the calculator in FIG. 3.

FIG. 5 is a flowchart illustrating a flow of processing of an audio analyzer of the processor of the calculator in FIG. 3.

FIG. 6 is a flowchart illustrating a flow of processing of an analyzer of the processor of the calculator in FIG. 3.

FIG. 7 is a distance waveform diagram based on typical distance information detected by the larynx portion displacement detector of the biological examination apparatus in FIG. 1.

FIG. 8 (a) is distance information based on detection data detected by a larynx portion displacement detector of the biological examination apparatus in FIG. 1 and a fitted movement waveform (fitting waveform) obtained from the distance information, and FIG. 8 (b) is a component waveform individually showing a movement trajectory over time in each of the up-and-down direction and the back-and-forth direction of the thyroid cartilage.

FIG. 9 is a swallowing sound waveform diagram including an envelope based on typical audio information detected by a swallowing sound detector of the biological examination apparatus in FIG. 1.

FIG. 10 illustrates an example of a trajectory graph displayed based on two-dimensional trajectory data obtained by a processor of the biological examination apparatus in FIG. 1.

FIG. 11 (a) is a waveform diagram illustrating a conventional way of grasping a component in a distance waveform, and FIG. 11 (b) is a waveform diagram illustrating a way of grasping of a component in a distance waveform in the present invention.

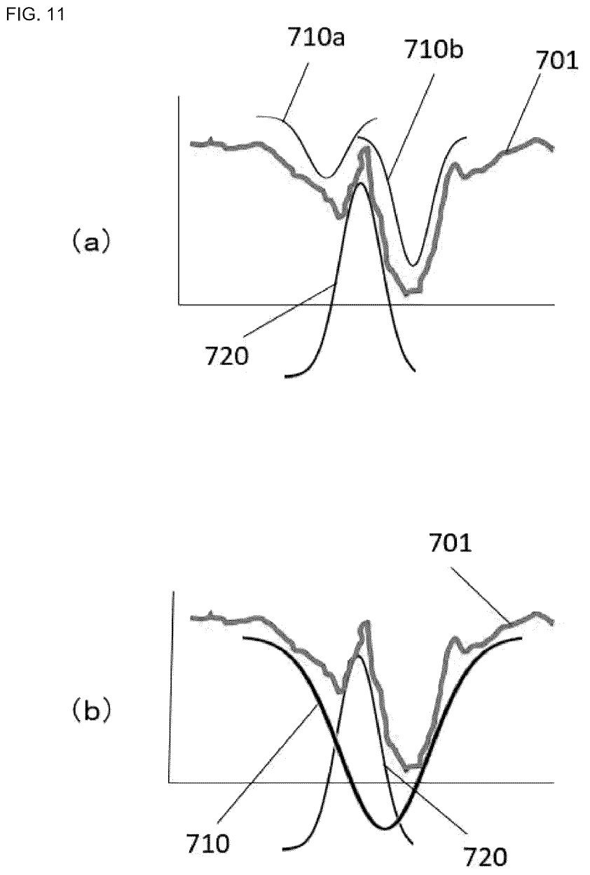

FIG. 12 is a schematic diagram showing an example of identification display that makes it possible to identify, on a trajectory graph, whether there is a peak of swallowing sound in the outbound path or the return path of the thyroid cartilage movement path.

FIG. 13 is a schematic diagram showing another example of identification display that makes it possible to identify, on a trajectory graph, whether there is a peak of swallowing sound in the outbound path or the return path of the thyroid cartilage movement path.

FIG. 14 is a diagram illustrating another example of reference information indicating a transition direction of a trajectory graph.

### DESCRIPTION OF EMBODIMENTS

Hereinafter, an embodiment of the present invention will be described with reference to the drawings. In the present embodiment, by providing the following technologies, a contribution to medical development and realization of a healthy society will be made. This examination apparatus and analysis method will contribute to the “9. Establish the basis of industry and technological innovation” of the United Nations' Sustainable Development Goals (SDGs).

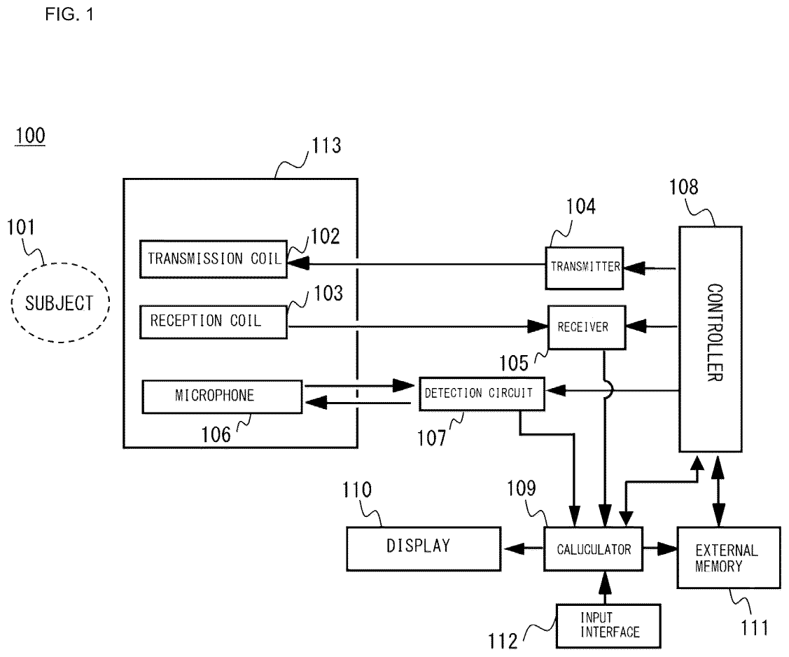

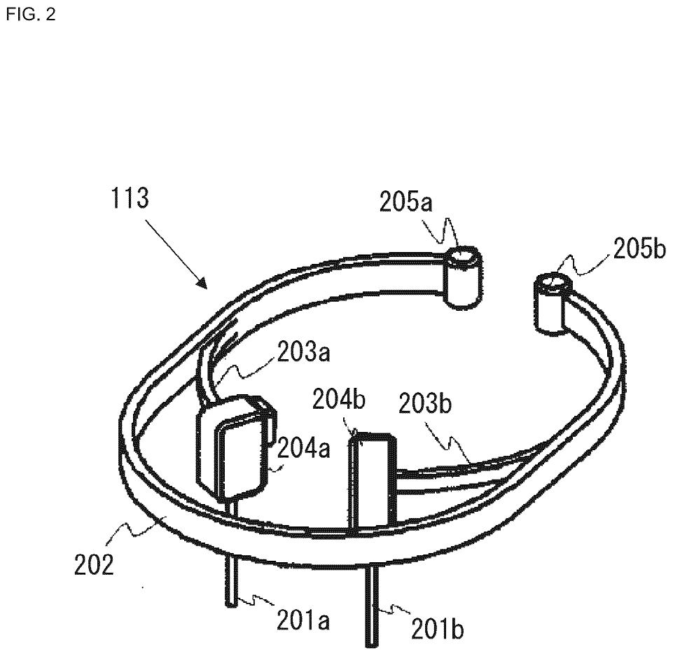

FIG. 1 is a functional block diagram illustrating a configuration example of a biological examination apparatus 100 according to an embodiment of the present invention. As illustrated in the drawing, the biological examination apparatus 100 includes a transmission coil 102 and a reception coil 103 as larynx portion displacement detectors that detect a change in a distance between two positions in the larynx (living body part around the thyroid cartilage) of a subject 101 caused by movements in the up-and-down and back-and-forth directions of the thyroid cartilage (common name: Adam's apple) at the time of swallowing of the subject 101; and a microphone 106 as a swallowing sound detector that detects a swallowing sound when the subject 101 swallows, and the coils 102 and 103 and the microphone 106 are held by a flexible holder 113 to be described later with reference to FIG. 2.

The transmission coil 102 and the reception coil 103 are arranged to face each other so that the thyroid cartilage is interposed from both sides, and the transmission coil 102 is connected to a transmitter 104 and the reception coil 103 is connected to a receiver 105. In addition, the microphone 106 is arranged in the vicinity of the thyroid cartilage of the subject 101, is electrically connected to a detection circuit 107 that detects the swallowing sound captured by the microphone 106 at the time of swallowing, and receives power supply or the like from the detection circuit 107 to operate. In addition, the microphone 106 is preferably a microphone using, for example, a piezoelectric element so as not to pick up ambient sounds other than the swallowing sound as much as possible, but may be a condenser type microphone or the like.

The biological examination apparatus 100 further includes a controller 108, a calculator 109, a display 110, an external memory 111, and an input interface 112. The controller 108 controls operations of the transmitter 104, the receiver 105, the detection circuit 107, the calculator 109, and the external memory 111, and controls power supply, signal transmission/reception timing, and the like. In addition, the calculator 109 is an information processing apparatus including a CPU, a memory, an internal memory, and the like, and performs various calculation processing. The control and calculation performed by the calculator 109 are realized by the CPU executing a predetermined program. However, a part of the calculation can also be realized by hardware such as an Application Specific Integrated Circuit (ASIC) or a Field Programmable Gate Array (FPGA). In addition, the display 110, the external memory 111, and the input interface 112 are electrically connected to the calculator 109.

In addition, the display 110 is an interface to display a measured waveform, analysis information of the calculator 109, and the like. The display 110 may be, for example, a liquid crystal display, an EL display, a plasma display, a CRT display, a projector, or the like, but is not limited thereto. In addition, the display 110 may be mounted on a tablet terminal, a head mounted display, a wearable device, or the like. In addition, notification of a specific function may be provided by an LED, a sound, and the like. In addition, the external memory 111 holds data used for various kinds of calculation processing executed by the calculator 109, data obtained by the calculation processing, conditions, parameters, and the like input through the input interface 112, together with the internal memory. In addition, the input interface 112 is an interface for the operator to input conditions and the like necessary for measurement and calculation processing performed in the present embodiment.

In such a configuration, a high frequency signal generated by the transmitter 104 is transmitted to the transmission coil 102, so that the transmission coil 102 emits a high frequency magnetic field, and accordingly, a signal received by the reception coil 103 is received by the receiver 105. In addition, the signal received by the receiver 105 is transmitted to the calculator 109 as an output voltage measurement value of the voltage between the coils. On the other hand, the swallowing sound captured by the microphone 106 is detected by the detection circuit 107, converted into a voltage signal, and input from the detection circuit 107 to the calculator 109 as an output voltage measurement value.

In FIG. 2, the flexible holder 113 that holds the transmission and reception coils 102 and 103 and the microphone 106 is shown. The flexible holder 113 is formed of any flexible material such as various resins. As illustrated in the drawing, the flexible holder 113 includes an approximately annular neck attachment member 202 to be attached to the neck of the subject 101 using the open end thereof and a pair of arc-shaped sensor holding members 203a and 203b located along approximately the same arc inside the neck attachment member 202. The neck attachment member 202 is integrally coupled so as to hold one end of each of the pair of sensor holding members 203a and 203b on both sides inside thereof, and the other ends of the sensor holding members 203a and 203b are opened to be located near the larynx of the subject 101. Then, sensors 204a and 204b are arranged at the other ends of the pair of sensor holding members 203a and 203b, respectively, and these sensors 204a and 204b are brought into contact with the larynx of the subject 101, and can follow the movement of swallowing (movement of the thyroid cartilage or the like) independently of the neck attachment member 202 together with the sensor holding members 203a and 203b located without being in contact with the neck of the subject 101.

The transmission coil 102 is arranged in one of the sensors 204a and 204b in a fixed state, the reception coil 103 is arranged in the other in a fixed state, and the microphone 106 is arranged in any one of the sensors 204a and 204b in a fixed state. In particular, in the present embodiment, the transmission coil 102 and the reception coil 103 are attached to the sensors 204a and 204b so as to be arranged in directions easily facing each other (close to the vertical direction of the neck surface of the subject 101), thereby enabling detection with a high signal-to-noise (SN) ratio. Therefore, the microphone 106 and the transmission coil 102 or the reception coil 103 can be arranged at positions approximately perpendicular to each other, and mixing of magnetic field noise generated from the microphone 106 into the transmission and/or reception coil 102 and 103 can be reduced. However, the corresponding positions of the transmission coil 102 and the reception coil 103 and the position perpendicular to the microphone are not limited to the described arrangement, and may be any position as long as detection with a sufficiently high SN ratio can be realized.

In addition, pressers 205a and 205b to be applied to the neck of the subject 101 are formed in a shape suitable for pressing, such as a cylindrical shape or a spherical shape, at opposing end portions forming the open end of the neck attachment member 202 (portions of the neck attachment member 202 located on the back side of the neck of the subject 101). The flexible holder 113 can be easily attached to the neck regardless of the size of the neck of the subject 101 by four pressing points including the two pressers 205a and 205b and the two sensors 204a and 204b provided at the other ends of the sensor holding members 203a and 203b. In addition, the transmission and reception coils 102 and 103 built in the sensors 204a and 204b and electric wirings 201a and 201b extending from the microphone 106 are electrically connected to the transmitter 104, the receiver 105, and the detection circuit 107 illustrated in FIG. 1, respectively.

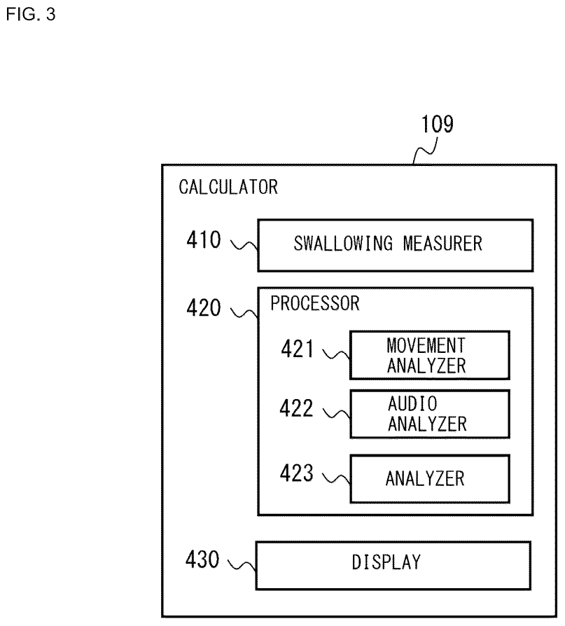

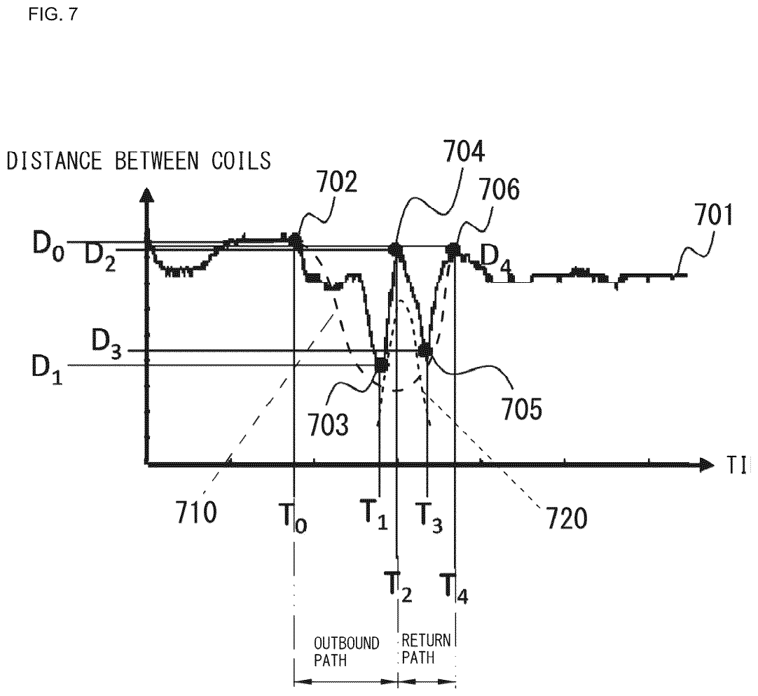

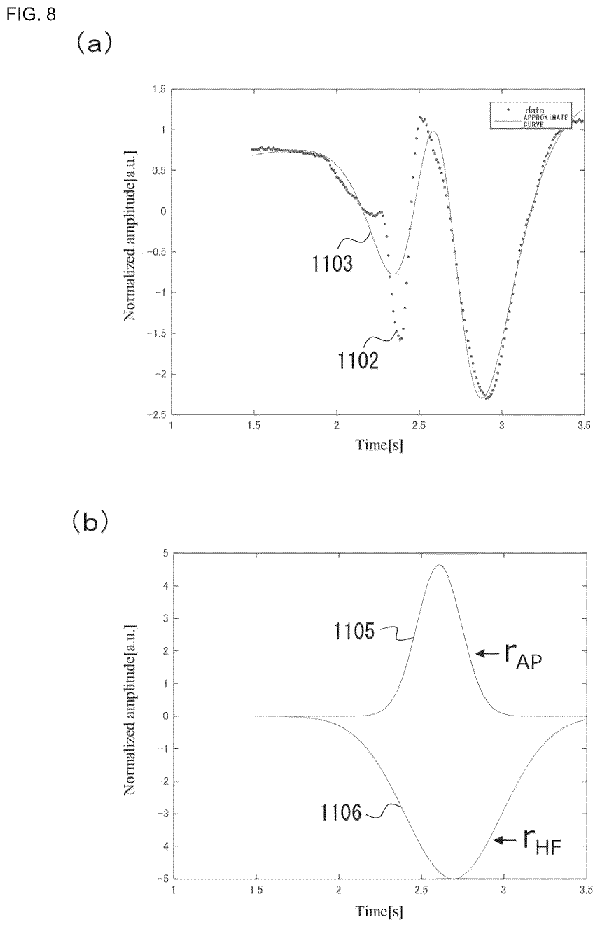

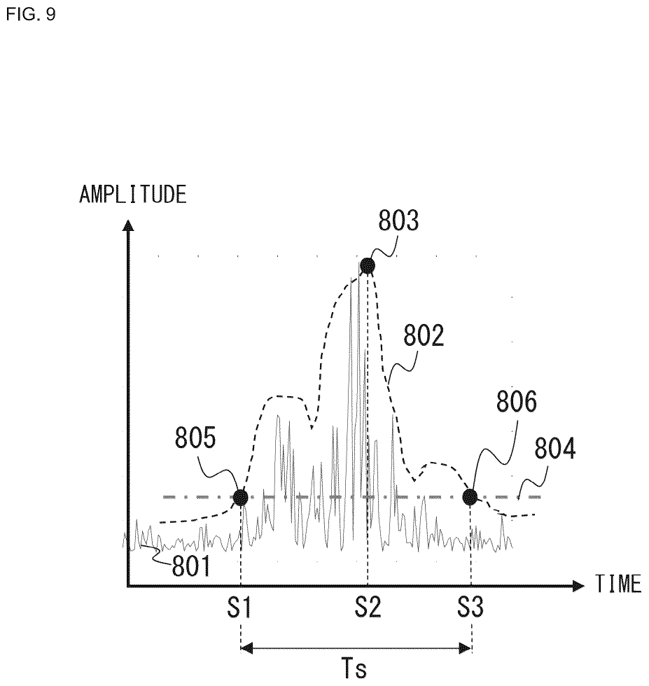

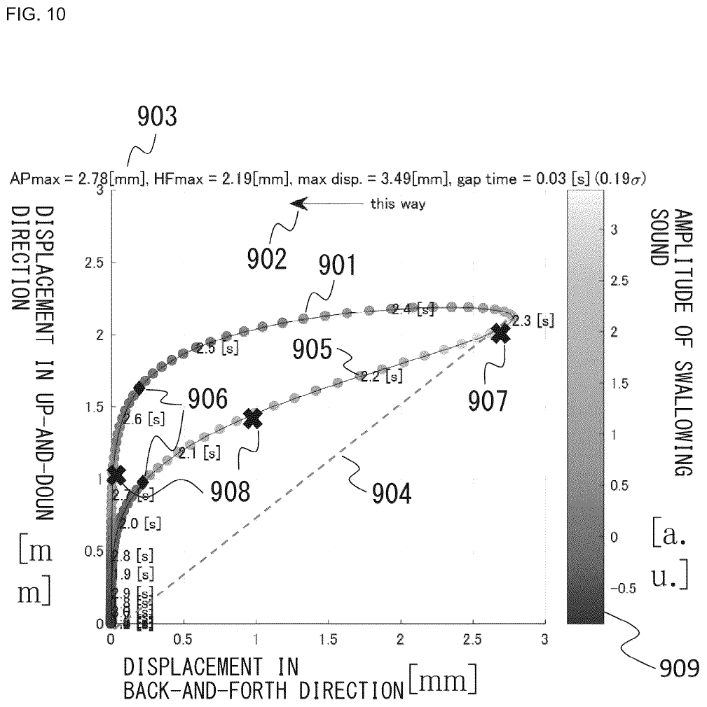

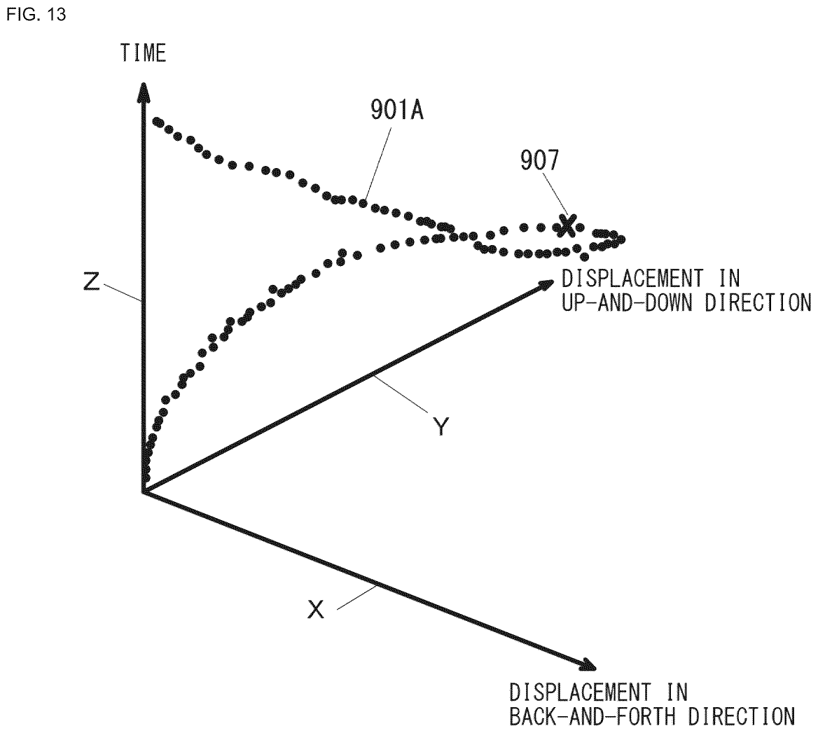

FIG. 3 is a functional block diagram of the calculator 109. As illustrated, the calculator 109 includes a swallowing measurer 410, a processor 420, and a display 430. The swallowing measurer 410 measures the swallowing movement and the swallowing sound using the transmission coil 102, the reception coil 103, the transmitter 104, the receiver 105, the microphone 106, the detection circuit 107, and the controller 108 described in association with FIG. 1 (larynx portion displacement detection step and swallowing sound detection step). In addition, the processor 420 includes a movement analyzer 421 that analyzes distance information, an audio analyzer 422 that analyzes a swallowing sound which is audio information, and an analyzer 423 that performs analysis by combining the distance information and the swallowing sound, and processes data measured by the swallowing measurer 410 (processing step). Specifically, as described later, the processor 420 obtains a fitting result (in the present embodiment, a fitted waveform 1103 illustrated in FIG. 8 (a) to be described later) obtained by fitting a model function (in the present embodiment, Equation (1) described below) that models the swallowing movement to distance information (in the present embodiment, data indicating a temporal change in the distance between the coils 102 and 103 arranged so that the thyroid cartilage of the subject 101 is interposed therebetween (a distance waveform 701 illustrated in FIG. 7 to be described later)) based on the detection data detected by the transmission and reception coils 102 and 103, extracts, from the fitting result, a back-and-forth movement component associated with the front and back movement of the thyroid cartilage (in the present embodiment, a back-and-forth movement component waveform 1105 shown in FIG. 8 (b), which will be described later, or a data value forming the back-and-forth movement component waveform 1105) and an up-and-down movement component associated with the up-and-down movement of the thyroid cartilage (in the present embodiment, an up-and-down movement component waveform 1106 shown in FIG. 8 (b), which will be described later, or a data value forming the up-and-down movement component waveform 1106), and generates two-dimensional trajectory data (in the present embodiment, data for forming a trajectory graph 901 illustrated in FIG. 10 to be described later) and three-dimensional trajectory data (in the present embodiment, data for forming a trajectory graph 901A illustrated in FIG. 13 to be described later) indicating the movement trajectory of the thyroid cartilage in its up-and-down and back-and-forth directions based on the extracted up-and-down movement components and back-and-forth movement components. In addition, as will be described later, the processor 420 generates identification display data that makes it possible to identify, on a trajectory graph 901, which of the outbound path of the movement path according to the elevation and the forward movement of the thyroid cartilage and the return path of the movement path according to the backward movement and the downward movement of the thyroid cartilage (see FIG. 7) has a peak of the swallowing sound in a series of up-and-down and back-and-forth movement paths of the thyroid cartilage at the time of swallowing, based on the detection data from the microphone 106. In addition, the processor 420 generates a swallowing sound waveform (in the present embodiment, a swallowing sound waveform 801 illustrated in FIG. 9 to be described later) indicating a temporal change in the amplitude of the swallowing sound based on the detection data detected by the microphone 106, and generates identification display data for identifying and displaying a plot of each trajectory data value on the trajectory graph according to the magnitude of the amplitude of the swallowing sound by temporally associating the swallowing sound waveform and the trajectory graph with each other. In addition, the display 430 displays information (data) measured and processed by the swallowing measurer 410 and the processor 420 on the display 110 (display step). In addition, the swallowing measurer 410, the processor 420, and the display 430 operate independently.

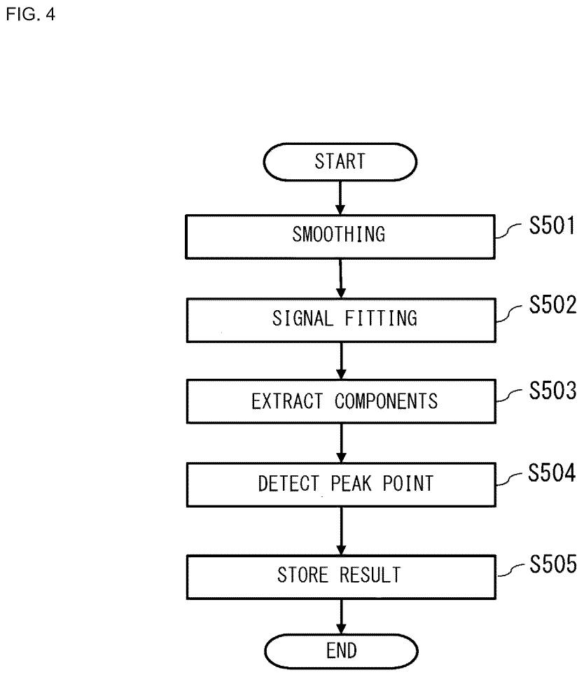

FIG. 4 illustrates a processing flow of the movement analyzer 421 of the processor 420 of the calculator 109 in FIG. 3. The movement analyzer 421 processes the detection data detected by the transmission and reception coils 102 and 103. Specifically, first, in step S501, the movement analyzer 421 performs smoothing on the data measured by the swallowing measurer 410. In particular, in the present embodiment, the smoothing is performed by piecewise polynomial approximation using the Savitzky-Golay filter. The smoothing in this case is performed by setting the number of windows and the degree of the polynomial to, for example, 5 and 51, respectively. In addition, the smoothing method may be, for example, simple moving average or the like, and the present invention is not limited to these methods.

Subsequently, in step S502, fitting is performed on the measurement signal smoothed in step S501. In this regard, FIG. 7 shows a typical example of a distance waveform 701 showing a temporal change in the distance between the transmission and reception coils 102 and 103, which is the distance between two positions in the larynx of the subject 101. Such a distance waveform 701 to be measured is a result of observing the two-dimensional movement (back-and-forth movement and up-and-down movement) of the thyroid cartilage (lingual bone) one-dimensionally (left and right), and has a W-shaped waveform shape as illustrated since the thyroid cartilage has a pyramidal shape. Specifically, the thyroid cartilage rises as the food bolus is fed into the esophagus from the start point (time T₀) 702 at which the subject 101 starts to swallow the food bolus by putting the food bolus into the mouth, so that the distance between the transmission and reception coils 102 and 103 narrows from D₀ to D₁, and the distance waveform 701 indicates the first valley (first lower limit peak value; time T₁) 703. In the feeding process, the epiglottis of the subject 101 moves downward, and the path from the nasal cavity to the airway is blocked. Thereafter, when the food bolus passes through the esophagus, the thyroid cartilage moves forward (in a direction in which the face of the subject faces) to open the esophagus, so that the distance between the transmission and reception coils 102 and 103 increases from D₁ to D₂, and the distance waveform 701 changes from the first valley portion 703 to a mountain portion (upper limit peak value; time T₂) 704. In this manner, a movement path (a section of the distance waveform 701 over time T₀ to time T₂) according to elevation and forward movement of the thyroid cartilage when the food bolus (or saliva) or the like is swallowed and passes through the esophagus forms an outbound path of the swallowing movement path. Then, when the food bolus completely passes through the esophagus (epiglottis) and is fed into the stomach, the thyroid cartilage also moves backward as the epiglottis moves upward, so that the distance between the transmission and reception coils 102 and 103 decreases from D₂ to D₃, and the distance waveform 701 changes from the mountain portion 704 to a second valley portion (second lower limit peak value; time T₃) 705. Thereafter, the thyroid cartilage descends so that the epiglottis and the thyroid cartilage return to their original positions, thereby increasing the distance between the transmission and reception coils 102 and 103 from D₃ to D₄, and the distance waveform 701 transitions from the second valley 705 to the end point (time T₄) 706. In this manner, a movement path (a section of the distance waveform 701 over time T₂ to time T₄) with backward movement and lowering of the thyroid cartilage when the food bolus (or saliva) or the like completely passes through the esophagus and is fed into the stomach forms a return path of the swallowing movement path.

As can be seen from the above, in such a distance waveform 701, a downwardly convex waveform component is generated in a series of movements from rising to falling of the thyroid cartilage, and an upwardly convex waveform component is generated in a series of movements from advancing to reversing of the thyroid cartilage. Therefore, in the present embodiment, the W-shaped distance waveform 701 is regarded as superposition of a gently downwardly convex waveform 710 (corresponding to the up-and-down movement component waveform 1106 illustrated in FIG. 8 (b)) and a sharp upwardly convex waveform 720 (corresponding to the back-and-forth movement component waveform 1105 illustrated in FIG. 8 (b)) as distinguished by a short broken line and a long broken line in FIG. 7, and is modeled as the following Equation (1).

$$\begin{matrix}[ Equation 1 ] & \\ y ( t ) = rAP ( t ) + rHF ( r ) + d ( t ) + e & ( 1 )\end{matrix}$$

Here, t represents time, y(t) represents a measured distance waveform, rAP(t) represents a component in the back-and-forth direction, rHF(t) represents a component in the up-and-down direction, d(t) represents a trend component generated from a body movement or the like (for example, an offset from an initial value caused by an individual difference such as a thickness of a neck), and e represents measurement noise.

In addition, in the present embodiment, the components rAP and rHF in the back-and-forth direction and the up-and-down direction are modeled by a normal distribution, and the trend component d(t) is modeled by a linear equation. However, these models may be autoregressive models or nonlinear models, and the present invention is not limited by these models. In such modeling of the present embodiment, each component is obtained by parameter fitting using a mathematical optimization method. In the present embodiment, parameter fitting is performed using the nonlinear least squares method, but the present invention is not limited thereto. In addition, when parameter fitting is performed, for example, a constraint that the variance value of rAP is smaller than the variance value of rHF may be provided.

FIG. 8 (a) illustrates a fitting result of the model function (Equation (1)) using such normal distribution. In the drawing, a waveform 1102 formed by a data value represented by a dot corresponds to the distance waveform 701 illustrated in FIG. 7, and a waveform 1103 represented by a solid line is a movement waveform (fitting waveform) obtained by fitting a model function to distance information forming the waveform 1102. Here, the horizontal axis is time, and the vertical axis is normalized amplitude based on the distance between the coils illustrated in FIG. 7.

After the signal fitting step S502 described above ends, parameters are extracted from the fitted model function in step S503. In the present embodiment, since the movement in the back-and-forth direction and the up-and-down direction of the thyroid cartilage is modeled with independent normal distributions, “amplitude”, “average value”, and “variance” of each of these movements are extracted in step S503. In addition, the “amplitude” corresponds to the magnitude of the movement of the thyroid cartilage, the “average value” corresponds to the time when the movement occurred, and the “variance” corresponds to the duration of the movement.

In this regard, FIG. 8 (b) illustrates waveforms (upwardly convex back-and-forth movement component waveform 1105 and downwardly convex up-and-down movement component waveform 1106) obtained by individually extracting and displaying only components in the back-and-forth direction and the up-and-down direction of the thyroid cartilage from the movement waveform (fitting waveform) 1103 illustrated in FIG. 8 (a). As described above, the processor 420 including the movement analyzer 421 of the biological examination apparatus 100 of the present embodiment can generate two-dimensional trajectory data individually indicating the temporary movement trajectory in each of the up-and-down direction and the back-and-forth direction of the thyroid cartilage based on the up-and-down movement component and the back-and-forth movement component.

After the component extraction step S503 ends, in step S504, feature points of the W-shaped waveform, that is, feature points corresponding to the peak points 702 to 706 (data values of D₀ to D₄ and T₀ to T₄) on the distance waveform 701 in FIG. 7 are extracted from the waveform reconstructed using the parameters extracted in step S503. Specifically, in the present embodiment, since the measurement signal is modeled and the components are separated as in Equation (1), the feature points are easily extracted without considering noise and a trend component. More specifically, as an example, T₂ is acquired as an average value of rAP, T₁ and T₃ are acquired as times indicating minimum values before and after T₂, respectively, and T₀ and T₄ are acquired as times at points advanced from the average value of rHF by variance values in negative and positive directions, respectively. Then, D₀ to D₄ are acquired as values corresponding to the times T₀ and T₄, respectively.

After the peak value detection step S504 ends, in step S505, the waveforms, the parameters, the feature points, and the like calculated in steps S501 to S504 described above are stored in the internal memory and/or the external memory 111 of the calculator 109. In addition, the above steps S501 to S505 may be performed during the measurement of the swallowing movement and the swallowing sound by the swallowing measurer 410, or may be performed a plurality of times.

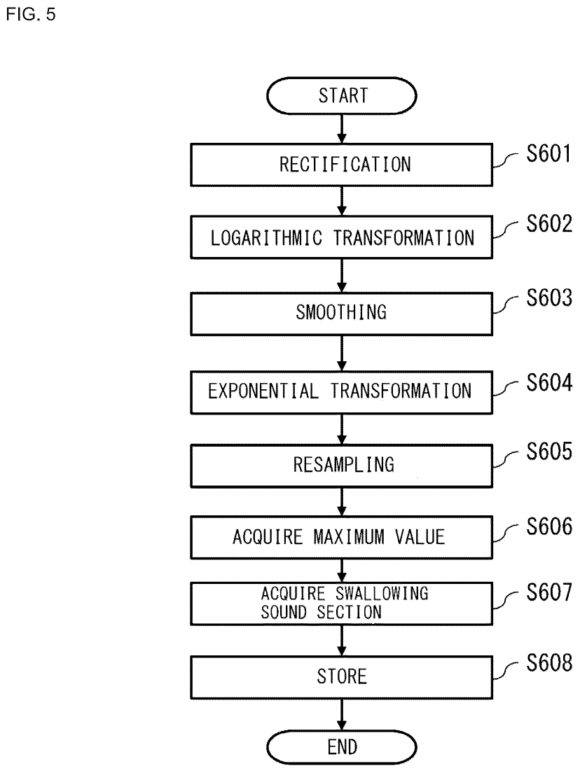

FIG. 5 illustrates a processing flow of the audio analyzer 422 of the processor 420 of the calculator 109 in FIG. 3. As illustrated, in step S601, rectification processing is performed on audio information (generally, an audio signal including both positive and negative values) measured from the microphone 106 through the swallowing measurer 410. Here, the rectification processing refers to processing for taking an absolute value to convert a negative value into a positive value. FIG. 9 illustrates a swallowing sound waveform 801 obtained by rectifying typical audio information.

In step S602, the signal subjected to the rectification processing obtained in step S601 is subjected to logarithmic transformation. This processing can reduce the influence of the spike-like signal mixed in the swallowing sound.

In step S603, smoothing is performed on the logarithmically transformed signal obtained in step S602. In particular, in the present embodiment, the smoothing processing is performed using the moving average, and the window width of the moving average is set to 400 points. In addition, the present invention is not limited by this smoothing method.

In step S604, exponential transformation is performed on the smoothing signal obtained in step S603. As a result, a waveform indicating the envelope of the initially measured audio information can be obtained. In FIG. 9, an envelope 802 obtained from such typical audio information (swallowing sound waveform 801) is indicated by a broken line.

In step S605, the envelope signal obtained in step S604 is resampled. Specifically, in the present embodiment, since the sampling frequencies of the audio information and the distance information in the swallowing measurer 410 illustrated in FIG. 3 are 4000 Hz and 100 Hz, respectively, processing for resampling the envelope signal to 1/40 to match the sampling frequency of the distance information is performed.

In step S606, a maximum value as a feature point is obtained for the resampled envelope signal obtained in step S605. This is because the section in which the maximum amplitude is obtained in the swallowing sound signal (swallowing sound waveform 801) is considered to indicate the flow of the ingested matter, and is an important feature of the swallowing sound. Therefore, in step S606, time S2 corresponding to the peak point 803 indicating the maximum amplitude with respect to the envelope 802 illustrated in FIG. 9 is acquired.

In step S607, the swallowing sound section of the resampled envelope signal obtained in step S605 is obtained. That is, in the envelope 802, the times at both ends of the swallowing sound section are acquired in order to obtain the time section Ts in which the swallowing sound occurs. Specifically, an amplitude threshold value 804 indicated by an alternate long and short dash line in FIG. 9 is set, and times S₁ and S₃ respectively corresponding to points crossing the threshold value 804 downward as viewed from the maximum value (peak point 803) obtained in step S606, that is, a temporally early start point 805 and a temporally late end point 806 are acquired as feature points. In the present embodiment, a value obtained by adding the normalized median absolute deviation to the median is used as the threshold value 804. In addition, the present invention is not limited by the method of setting the threshold value 804, and a value obtained by adding a standard deviation to an average value or the like may be used.

Finally, in step S608, the waveforms, the feature amounts, and the like calculated in steps S601 to S607 described above are stored in the internal memory and/or the external memory 111 of the calculator 109. In addition, the above steps S601 to S608 may be performed during the measurement of the swallowing movement and the swallowing sound by the swallowing measurer 410, or may be performed a plurality of times.

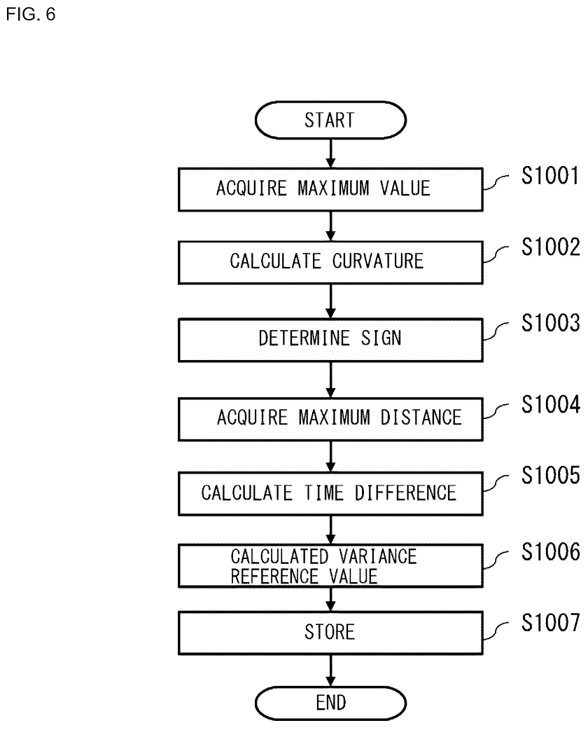

FIG. 6 illustrates a processing flow of the analyzer 423 of the processor 420 of the calculator 109 in FIG. 3. As illustrated, in step S1001, the maximum displacement (maximum value) in the back-and-forth direction and the up-and-down direction of the movement waveform 1103 (or the distance waveform 701) which is the fitted waveform is calculated.

In step S1002, the signed curvature at each point on the trajectory graph 901 described in detail below with reference to FIG. 10 is calculated. In step S1002, the time progress direction (transition direction) of the trajectory graph 901 is extracted, and the signed curvature at each point on the trajectory graph 901 is calculated in order to extract the point having the maximum displacement.

In step S1003, a sign is acquired in the signed curvature obtained in step S1002. Specifically, in the trajectory graph 901, since the amplitude of the curvature is maximized at the point farthest from the coordinate origin, the sign of the point having the maximum curvature is acquired after the curvature at each point on the trajectory graph 901 is calculated. By determining the sign such that the counterclockwise direction is positive and the clockwise direction is negative as the coordinate system, the time progress direction is uniquely obtained. In addition, the factor that determines whether the sign is positive or negative is the magnitude of the average value of the component rAP in the back-and-forth direction and the component rHF in the up-down direction. In a trajectory graph 901 in FIG. 10 described later in which the time progress direction is the counterclockwise direction, it is indicated that the average value (that is, the time for taking the maximum value) of the displacement in the back-and-forth direction is earlier than that in the up-down direction.

In step S1004, the geometric distance from the coordinate origin of the point at which the maximum value of the signed curvature calculated in step S1002 is obtained is acquired. In the trajectory graph 901, since the amplitude of the curvature is maximized at the point farthest from the coordinate origin, the geometric distance from the point at which the amplitude of the curvature is maximized to the coordinate origin is calculated. As a result, it is possible to acquire a time point (time) at which the displacement is the largest when the components in the up-and-down direction and the back-and-forth direction of the thyroid cartilage are combined.

In step S1005, a time difference between the time at which the maximum value of the audio information is obtained and the time at which the maximum value of the distance information in the back-and-forth direction is obtained. This is particularly because the time difference taking the maximum value is an important parameter in characterizing the swallowing state. In the present embodiment, as can be seen from the display form of the trajectory graph 901 to be described later, this parameter can be not only visually grasped but also displayed as a quantitative value. In addition, the present invention is not limited by these quantitative values, and for example, the area of the region surrounded by the trajectory graph may be displayed as the feature amount.

In step S1006, a ratio (a ratio of the time difference to the variance value) of the time difference obtained in step S1005 based on the variance value of the model (back-and-forth movement component waveform 1105 illustrated in FIG. 8 (b)) indicating the back-and-forth movement component of the distance information is acquired. Since the swallowing sound is generated at the timing when the thyroid cartilage advances in the healthy subject model, in step S1006, the ratio is calculated in order to display the degree of the deviation in the occurrence of the swallowing sound in the individual.

Finally, in step S1007, the waveforms, the feature amounts, and the like calculated in steps S1001 to S1006 described above are stored in the internal memory and/or the external memory 111 of the calculator 109. In addition, the above steps S1001 to S1007 may be performed during the measurement of the swallowing movement and the swallowing sound by the swallowing measurer 410, or may be performed a plurality of times.

Based on the processing steps described above, the processor 420 further generates, as an example, two-dimensional trajectory data simultaneously showing the movement in the up-and-down direction and the back-and-forth direction of the thyroid cartilage as one trajectory graph 901 (see FIG. 10) based on the above-described up-and-down movement component and the back-and-forth movement component. Specifically, such two-dimensional trajectory data is generated as coordinate data indicated on a coordinate plane defined by two coordinate axes perpendicular to each other. One of the coordinate axes corresponds to a trajectory data value of a back-and-forth movement component, and the other coordinate axis corresponds to a trajectory data value of an up-and-down movement component. More specifically, as illustrated in FIG. 10, based on the signal fitting (step S502 in FIG. 4) and the component extraction (step S503 in FIG. 4) by the movement analyzer 421 described above, the data value on the up-and-down movement component waveform 1106 and the data value on the back-and-forth movement component waveform 1105 described above are temporally associated with each other to be plotted with a trajectory data value of the back-and-forth movement component (displacement in the back-and-forth direction; normalized amplitude in the back-and-forth movement component waveform 1105) on the horizontal axis and a trajectory data value of the up-and-down movement component (up-and-down displacement; normalized amplitude in the up-and-down movement component waveform 1106) on the vertical axis. That is, the horizontal axis indicates the value of the normal distribution having the parameter extracted for rAP of Equation (1) in step S503 of FIG. 4, and the vertical axis indicates the value of the normal distribution having the parameter extracted for rHF of Equation (1).

Such a trajectory graph 901 illustrated in FIG. 10 is displayed on the display 110 through the display 430 of the calculator 109. In particular, in the present embodiment, plots of respective trajectory data values on the trajectory graph 901 are identified and displayed according to the magnitude of the amplitude of the swallowing sound, for example, displayed in different colors. In order to realize such identification display, the processor 420 generates the swallowing sound waveform 801 and the envelope 802 indicating a temporal change in the amplitude of the swallowing sound based on the detection data detected through the microphone 106 as described above, and generates identification display data for identifying and displaying the plot of each trajectory data value on the trajectory graph 901 according to the magnitude of the amplitude of the swallowing sound by temporally associating the swallowing sound waveform 801 or the envelope 802 with the trajectory graph 901. In the present embodiment in which color-coded display is performed in association with such identification display, a band graph 909 for reference indicating how the color changes with the magnitude of the swallowing sound amplitude value along the vertical axis is displayed adjacent to the trajectory graph 901. For example, here, the identification display form is formed such that the larger the amplitude of the swallowing sound, the more yellowish it is, and the smaller the amplitude, the more blue-colored it is. Alternatively, the identification display mode may be such that color coding is performed in black and white, and the color becomes lighter as the amplitude increases. In addition, the identification display form is not limited to this, and any display form may be used as long as the trajectory data values having different amplitudes of the swallowing sound can be identified by changing the size or shape of the plot (mark) of each trajectory data value according to the magnitude of the amplitude of the swallowing sound.

Such a trajectory graph 901 in which trajectory data values are plotted as a time-series scatter diagram displays movements in the back-and-forth direction and the up-and-down direction of the thyroid cartilage separately on the two coordinate axes so that the movement of the thyroid cartilage in swallowing can be grasped at a glance. In addition, by displaying the characteristic of the swallowing sound information in addition to the movement of the swallowing movement in one trajectory graph 901 as described above, it is possible to visually check at which time point the swallowing sound has occurred with respect to the movement of the thyroid cartilage. Therefore, it is possible not only to quantitatively grasp the swallowing movement but also to grasp the deviation of the swallowing sound from the normal state and the power of the swallowing sound at a glance.

In addition, various kinds of auxiliary information are added and displayed on the trajectory graph 901. For this purpose, in the present embodiment, the processor 420 generates supplementary display data for displaying supplementary information including the predetermined feature point associated with the movement waveform 1103 (or the distance waveform 701), the predetermined feature point associated with the swallowing sound waveform 801 (or the envelope 802), and the generation time of the trajectory data value plotted on the trajectory graph 901 to be superimposed on the trajectory graph 901, and also generates reference display data for displaying the reference information including the transition direction of the trajectory graph 901 and the predetermined feature amount calculated from the trajectory graph 901 together with the trajectory graph 901.

Specifically, with respect to such an auxiliary display, reference numeral 902 in FIG. 10 denotes an arrow indicating in which direction the trajectory has progressed (the transition direction of the trajectory graph 901). In the present embodiment, the trajectory starts from the coordinate origin, rotates counterclockwise, and then returns to the coordinate origin. In addition, 903 indicates a feature amount calculated from the trajectory graph 901. Specifically, the maximum amount of displacement in the back-and-forth direction, the maximum amount of displacement in the up-down direction, the maximum displacement from the coordinate origin indicated by 904, the time difference (o) of the time during which the movement information and the audio information each take the maximum value and the ratio of the time difference based on the variance value of the displacement (rAP) in the back-and-forth direction are indicated as feature amounts. These pieces of information are obtained by the processing by the analyzer 423 described above. As a method of displaying the feature amount, the feature amount may be displayed in the coordinate region of the trajectory graph 901 or may be displayed in another diagram instead of being displayed above the coordinate region of the trajectory graph 901 as in the present embodiment, and the present invention is not limited thereto.

In FIG. 10, reference numeral 905 denotes an occurrence time of a trajectory data value plotted on the trajectory graph 901, and is displayed every 0.1 seconds in the present embodiment. Reference numeral 906 denotes a peak point in the distance information obtained in step S504 in FIG. 4. In addition, 907 indicates a time point at which the maximum value of the audio information obtained in step S606 of FIG. 5 is obtained. With this display, the time difference between the time point indicating the maximum value of the audio information and the time point indicating the maximum value of the component in the back-and-forth direction of the thyroid cartilage in the distance information can be checked in the drawing. In addition, 908 indicates the start point 805 and the end point 806 (see FIG. 9) of the audio information obtained in step S607 of FIG. 5.

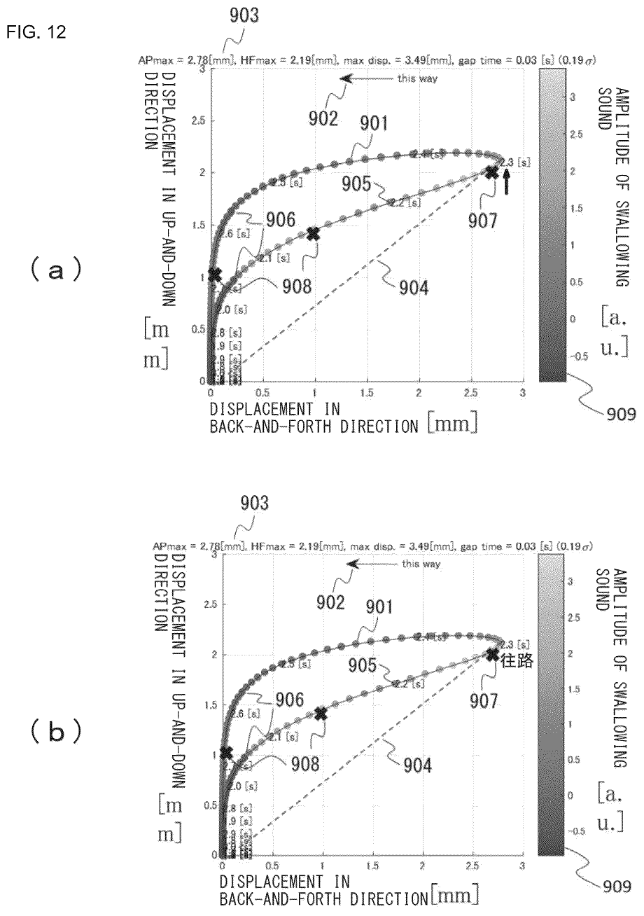

In addition, in the present embodiment, in addition to the display form illustrated in FIG. 10, it is possible to identify, on the trajectory graph 901, which of the outbound path of the movement path according to the elevation and the forward movement of the thyroid cartilage (see FIG. 7) and the return path of the movement path according to the backward movement and the downward movement of the thyroid cartilage (see FIG. 7) has the peak 907 of the swallowing sound in a series of up-and-down and back-and-forth movement paths of the thyroid cartilage at the time of swallowing. For this purpose, the processor (processing step) 420 generates identification display data that makes it possible to identify, on the trajectory graph 901, which of the outbound path of the movement path according to the elevation and the forward movement of the thyroid cartilage and the return path of the movement path according to the backward movement and the downward movement of the thyroid cartilage has a peak of the swallowing sound in a series of up-and-down and back-and-forth movement paths of the thyroid cartilage at the time of swallowing, based on the detection data from the microphone 106. Specifically, for example, when a signal to make a request for such a display form is input through the input interface 112, as illustrated in FIG. 12 (a), for example, an upward arrow (in the case of the return path, for example, a downward arrow) indicating the outbound path is displayed adjacent to the position of the peak 907 of the swallowing sound. In this case, an explanatory display of an arrow regarding the outbound path/the return path is displayed adjacent to the trajectory graph 901. Needless to say, an icon or the like may be used instead of the arrow. Alternatively, the display form of FIG. 12 may be automatically displayed instead of the display form of FIG. 10 without requiring input from the outside.

In addition, as illustrated in FIG. 12 (b), a text (character) indicating the outbound path/the return path may be displayed adjacent to the position of the peak 907 of the swallowing sound. In addition, the trajectory graph 901 may be displayed in different colors for the outbound path and the return path (plots of trajectory data values of distance information may be displayed in different colors) so that it is possible to grasp at a glance whether the peak 907 of the swallowing sound is present in the outbound path or the return path of the thyroid cartilage movement path. In this case, for example, the color-coded display of the swallowing sound amplitude and the color-coded display of the outbound path/the return path may be switched by a switching signal input through the input interface 112 so as not to overlap the color-coded display of the amplitude of the swallowing sound. In addition, by such switching, instead of or in addition to the reference band graph 909 indicating the magnitude of the swallowing sound amplitude value, a reference display (not illustrated) clearly indicating the color-coding state of the outbound path/the return path may be displayed adjacent to the trajectory graph 901.

Further, instead of the two-dimensional trajectory graph 901 illustrated in FIGS. 10 and 12, a three-dimensional trajectory graph 901A illustrated in FIG. 13 may be displayed on the display 110 through the display 430. For this purpose, the processor (processing step) 420 extracts an up-and-down movement component associated with the up-and-down movement of the thyroid cartilage and a back-and-forth movement component associated with the back-and-forth movement of the thyroid cartilage from a fitting result obtained by fitting a model function modeling the swallowing movement to distance information based on detection data detected by the transmission and reception coils 102 and 103, and generates three-dimensional trajectory data simultaneously indicating movements in the up-and-down direction and the back-and-forth direction of the thyroid cartilage in one trajectory graph based on the extracted up-and-down movement component and the back-and-forth movement component. In this case, as illustrated in FIG. 13, the three-dimensional trajectory data is generated as coordinate data indicated in a coordinate space defined by three coordinate axes perpendicular to each other, and the three coordinate axes include a coordinate axis X corresponding to the trajectory data value of the back-and-forth movement component, a coordinate axis Y corresponding to the trajectory data value of the up-and-down movement component, and a coordinate axis Z indicating the swallowing movement time. In addition, in order to simplify the drawing, auxiliary and reference displays of feature points and the like are omitted in FIG. 13, but it goes without saying that various feature point displays and reference displays are added in FIG. 13 as illustrated in FIGS. 10 and 12.

When such trajectory data based on distance information is indicated in three dimensions, overlapping of dots of data values can be avoided, and for example, the peak position of the swallowing sound in the thyroid cartilage movement path at the time of swallowing can also be clearly grasped at a glance. Also in this case, text or symbols indicating the outbound path/the return path may be displayed adjacent to the position of the peak 907 of the swallowing sound, or the trajectory graph 901 may be displayed in different colors for the outbound path and the return path.

As described above, according to the display form illustrated in FIGS. 12 and 13, the peak position of the swallowing sound in the thyroid cartilage movement path at the time of swallowing can be grasped at a glance, and the swallowing movement can be accurately evaluated. In addition, such identification display is useful particularly in a case where it is difficult to grasp which one of the outbound path and the return path of the thyroid cartilage movement path has a peak of the swallowing sound because dots of data plotted on the graph overlap each other, such as a case where the peak position of the swallowing sound is a position close to the coordinate origin in the two-dimensional trajectory graph display (display form of FIG. 10) based on the two-dimensional trajectory data.

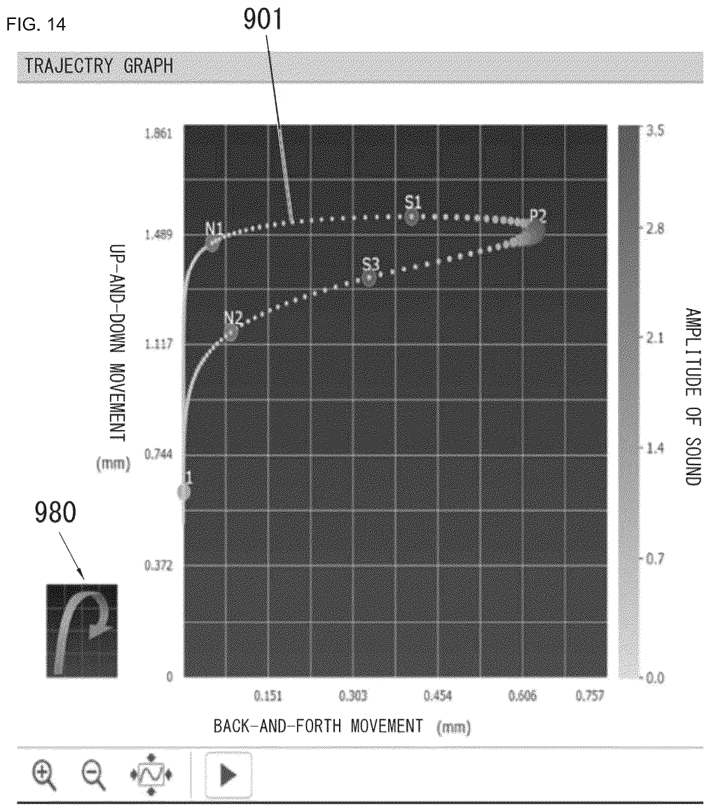

FIG. 14 illustrates a two-dimensional trajectory graph 901 similar to FIG. 10, but here, instead of the arrow 902 of FIG. 10, reference information indicating a transition direction of the trajectory graph 901 is displayed as an icon 980 together with the trajectory graph 901. Therefore, the processor 420 generates reference display data for displaying such an icon 980 together with the trajectory graph 901. In this case, S1 and S3 on the trajectory graph 901 are times respectively corresponding to the temporally early start point 805 and the temporally late end point 806 in the swallowing sound (see FIG. 9), P2 corresponds to the upper limit peak value of the swallowing sound, N1 corresponds to the start point of the upward movement of the thyroid cartilage in the swallowing movement, and N2 corresponds to the start point of the backward movement of the thyroid cartilage in the swallowing movement. Alternatively, the transition direction of the trajectory graph 901 may be grasped by displaying the trajectory graph 901 as a moving image (animation) without displaying the transition direction of the trajectory graph as an arrow or an icon.

In addition, the present invention is not limited to the above-described embodiments, and various modifications can be made without departing from the gist thereof. For example, in the above-described embodiments, the present invention is applied to the movement of the thyroid cartilage, but the present invention can also be applied to the examination of the movement of a body part other than the thyroid cartilage. That is, the present invention can also be applied to analysis of movement of a part other than the larynx portion as long as the part is a body part that makes a movement (back-and-forth and up-and-down movements) similar to that of the thyroid cartilage (lingual bone). Specifically, the present invention can be applied to any body part as long as a change in distance detected by a predetermined detector can be analyzed by decomposing the change in distance into movements in a plurality of directions. In addition, the biological examination apparatus of the present invention may not include the larynx portion displacement detector, the swallowing sound detector, and the display as described above. That is, the biological examination apparatus, the larynx portion displacement detector, the swallowing sound detector, and the display may be configured as separate systems. In addition, the processing of each apparatus described in the present embodiment may be realized by any of software, hardware, and a combination of software and hardware. The program forming the software may be stored in, for example, a non-transitory computer readable medium. In addition, the program may be distributed through a network, for example. In addition, within the scope not departing from the gist of the present invention, a part or all of the above-described embodiments may be combined, or a part of the configuration may be omitted from one of the above-described embodiments.

### REFERENCE SIGNS LIST

100 biological examination apparatus
102 transmission coil (larynx portion displacement detector)
103 reception coil (larynx portion displacement detector)
106 microphone (swallowing sound detector)
420 processor
430 display

1. A biological examination apparatus, comprising:
a larynx portion displacement detector that detects a change in a distance between two positions in a larynx portion of a subject caused by movement of a thyroid cartilage in an up-and-down direction and a back-and-forth direction at the time of swallowing;
a swallowing sound detector that detects a swallowing sound when the subject swallows; and
a processor that processes detection data from the larynx portion displacement detector and the swallowing sound detector,
wherein the processor
extracts an up-and-down movement component associated with an up-and-down movement of the thyroid cartilage and a back-and-forth movement component associated with a back-and-forth movement of the thyroid cartilage from a fitting result of fitting a model function modeling a swallowing movement to distance information based on the detection data detected by the larynx portion displacement detector and generates two-dimensional or three-dimensional trajectory data, which indicates the movement of the thyroid cartilage in the up-and-down direction and the back-and-forth direction simultaneously in one trajectory graph, based on the extracted up-and-down movement component and back-and-forth movement component, and
generates identification display data that makes it possible to identify, on the trajectory graph, which of an outbound path of a movement path according to elevation and forward movement of the thyroid cartilage and a return path of the movement path according to a backward movement and a downward movement of the thyroid cartilage has a peak of swallowing sound in a series of up-and-down and back-and-forth movement paths of the thyroid cartilage at the time of swallowing, based on the detection data from the swallowing sound detector.

2. The biological examination apparatus according to claim 1, wherein the processor generates reference display data for displaying reference information indicating a transition direction of the trajectory graph together with the trajectory graph.

3. The biological examination apparatus according to claim 1, wherein the two-dimensional trajectory data is generated as coordinate data indicated on a coordinate plane defined by two coordinate axes perpendicular to each other, one of the coordinate axes corresponding to a trajectory data value of the back-and-forth movement component and the other coordinate axis corresponding to a trajectory data value of the up-and-down movement component.

4. The biological examination apparatus according to claim 1, wherein the three-dimensional trajectory data is generated as coordinate data indicated in a coordinate space defined by three coordinate axes perpendicular to each other, the three coordinate axes including a coordinate axis corresponding to a trajectory data value of the back-and-forth movement component, a coordinate axis corresponding to a trajectory data value of the up-and-down movement component, and a coordinate axis indicating a swallowing movement time.

5. The biological examination apparatus according to claim 1, wherein the processor generates a swallowing sound waveform indicating a temporal change in amplitude of the swallowing sound based on the detection data from the swallowing sound detector, and generates identification display data for identifying and displaying a plot of each trajectory data value on the trajectory graph according to a magnitude of the amplitude of the swallowing sound by temporally associating the swallowing sound waveform and the trajectory graph with each other.

6. The biological examination apparatus according to claim 5, wherein the processor generates supplementary display data for displaying supplementary information, which includes at least one of a predetermined feature point associated with the fitting result, a predetermined feature point associated with the swallowing sound waveform, and an occurrence time of a trajectory data value plotted on the trajectory graph, to be superimposed on the trajectory graph.

7. The biological examination apparatus according to claim 1, wherein the processor generates reference display data for displaying reference information indicating a predetermined feature amount calculated from the trajectory graph together with the trajectory graph.

8. The biological examination apparatus according to claim 1, wherein the larynx portion displacement detector includes a transmission coil and a reception coil that are arranged so as to interpose the thyroid cartilage from both sides and transmit and receive a high frequency signal.

9. A biological information analysis method, comprising:
a larynx portion displacement detection step for detecting a change in a distance between two positions in a larynx portion of a subject caused by movement of a thyroid cartilage in an up-and-down direction and a back-and-forth direction at the time of swallowing;
a swallowing sound detection step for detecting a swallowing sound when the subject swallows as biological information;
a processing step for processing detection data detected in the larynx portion displacement detection step and the swallowing sound detection step; and
a display step for displaying data processed in the processing step,
wherein, in the processing step,
an up-and-down movement component associated with an up-and-down movement of the thyroid cartilage and a back-and-forth movement component associated with a back-and-forth movement of the thyroid cartilage are extracted from a fitting result of fitting a model function modeling a swallowing movement to distance information based on the detection data detected by the larynx portion displacement detector, and two-dimensional or three-dimensional trajectory data indicating the movement of the thyroid cartilage in the up-and-down direction and the back-and-forth direction simultaneously in one trajectory graph is generated based on the extracted up-and-down movement component and back-and-forth movement component, and
identification display data that makes it possible to identify, on the trajectory graph, which of an outbound path of a movement path according to elevation and forward movement of the thyroid cartilage and a return path of the movement path according to a backward movement and a downward movement of the thyroid cartilage has a peak of swallowing sound in a series of up-and-down and back-and-forth movement paths of the thyroid cartilage at the time of swallowing is generated based on the detection data from the swallowing sound detector.

10. The biological information analysis method according to claim 9, wherein, in the processing step, reference display data for displaying reference information indicating a transition direction of the trajectory graph together with the trajectory graph is generated.

11. A computer program
causing a computer to execute:
a larynx portion displacement detection step for detecting a change in a distance between two positions in a larynx portion of a subject caused by movement of a thyroid cartilage in an up-and-down direction and a back-and-forth direction at the time of swallowing;
a swallowing sound detection step for detecting a swallowing sound when the subject swallows as biological information;
a processing step for processing detection data detected in the larynx portion displacement detection step and the swallowing sound detection step; and
a display step for displaying data processed in the processing step,
wherein, in the processing step,
an up-and-down movement component associated with an up-and-down movement of the thyroid cartilage and a back-and-forth movement component associated with a back-and-forth movement of the thyroid cartilage are extracted from a fitting result of fitting a model function modeling a swallowing movement to distance information based on the detection data detected by the larynx portion displacement detector, and two-dimensional or three-dimensional trajectory data indicating the movement of the thyroid cartilage in the up-and-down direction and the back-and-forth direction simultaneously in one trajectory graph is generated based on the extracted up-and-down movement component and back-and-forth movement component, and
identification display data that makes it possible to identify, on the trajectory graph, which of an outbound path of a movement path according to elevation and forward movement of the thyroid cartilage and a return path of the movement path according to a backward movement and a downward movement of the thyroid cartilage has a peak of swallowing sound in a series of up-and-down and back-and-forth movement paths of the thyroid cartilage at the time of swallowing is generated based on the detection data from the swallowing sound detector.

12. The computer program according to claim 11, wherein, in the processing step, reference display data for displaying reference information indicating a transition direction of the trajectory graph together with the trajectory graph is generated.
